(19) United States
(12) Patent Application Publication
(10) Pub. No.: US 2026/0284341 A1
(43) Pub. Date: Sep. 24, 2026

(54) BREATHING APPARATUS AND METHOD FOR CONTROLLING A BREATHING APPARATUS

(71) Applicant: IMTMEDICAL AG, Buchs SG (CH)

(72) Inventors: Harri Friberg, Mauren (LI); Jakob Däscher, Fläsch (CH)

(21) Appl. No.: 19/690,994

(22) Filed: May 28, 2026

Related U.S. Application Data
(63) Continuation of application No. 16/620,773, filed on Jun. 29, 2018.

(30) Foreign Application Priority Data
Jun. 30, 2017 (CH) 00857/17

(51) Int. Cl. A61M 16/00; G16H 20/40

(52) U.S. Cl. CPC A61M 16/022; A61M 16/0003; G16H 20/40; A61M 2016/0027; A61M 2205/3576; A61M 2205/505; A61M 2205/583

(57) ABSTRACT

A breathing apparatus (15), is connected to a sensor system (30) and to a control system (24), wherein the sensor system (30) is designed for capturing at least two items of measurement data (31) and for transmitting the captured measurement data (31) to the breathing apparatus (15) or the control logic module (25). The control system (24) is further connected to at least one indicating device (35), wherein the at least one indicating device (35) has a configurable screen (33). The control system (24) is designed for the presentation of indicated data (62, 65) based on the captured measurement data (31), which may be displayed on a first graphical unit (29) on the at least one indicating device (35). The invention furthermore relates to a method for controlling a breathing apparatus (15).

### CROSS-REFERENCE TO RELATED APPLICATIONS

This is a continuation of U.S. patent application Ser. No. 16/620,773 (filed 9 Dec. 2019), which was the National Stage of International Patent Application PCT/IB2018/054852 (filed 29 Jun. 2018), which claims the benefit of Switzerland Patent Application 00857/17 (filed 30 Jun. 2017). Each of these priority applications is hereby incorporated by reference herein in its entirety.

### FIELD OF INVENTION

The invention relates to a ventilator as well as to a method for controlling a ventilator.

### BACKGROUND

Ventilators are used both in stationary situations (for example in the clinic or domestic environment) and also in mobile situations (for example with the emergency services). In this regard, it is important for the ventilators to operate reliably and without malfunctions.

A further requirement for ventilators of this type is ease of operation. If an operator makes an error, this could have disastrous consequences for a patient being ventilated using the ventilator.

WO 02/071933 A2 discloses a ventilator which is connected to a sensor system as well as to a control system, wherein the control system is connected to a display means. The sensor system acquires measurement data and transmits this to the ventilator. The control system provides display data on the basis of the acquired measurement data, which can be displayed on the display means as an animated graphics unit.

EP 1 984 805 B1 discloses a ventilator in which a graphical element in the form of a lung is shown on a display means. The volume change of the ventilated lung which occurs at each breath is shown as an animated change in the size of the lung shape.

Thus, the objective of the present invention is to provide a ventilator which is easy for the operator to operate and is safe to use. The acquired measurement data should in this regard be made available to the operator in both a qualitative and quantitative manner in an optimized form. A further objective of the invention is to provide a method for controlling a ventilator of this type.

### SUMMARY

The objective is achieved by means of the features of the independent claims. Advantageous further refinements are shown in the figures and in the dependent patent claims.

In the application below, the expression “or” linking two terms is used with the meaning “and/or”. This means that it should be understood that the first term “or” the second term could be meant by this, but also that it includes the first term “and” the second term.

The ventilator in accordance with the invention is connected to a sensor system as well as to a control system. The control system may be a component of the ventilator.

The sensor system may also be a component of the ventilator. The sensor system is configured to acquire at least two items of measurement data as well as to transmit the acquired measurement data to the ventilator or to the control logic module.

As an example, the sensor system comprises at least two measuring sensors, wherein each measuring sensor acquires measurement data from one origin. The sensors are advantageously configured in different manners and acquire different measurement data. Alternatively, the sensor system comprises just one measuring sensor which acquires at least two items of measurement data from different origins.

The control system is linked to a display means which comprises a configurable screen. The term “configurable screen” in this context means a screen which not only allows the depicted individual components to be discerned, but also allows the totality of all of the depicted components shown and their dispositions to be observed. In this regard, the configurable screen acquires measurement data either autonomously or with the aid of a graphic logic module or a control logic module and transforms these into geometrical elements which can then be displayed. Furthermore, the configurable screen is capable of changing existing elements (geometrical and/or graphical) in a graphics unit and of transforming modifications in the graphics unit into parameters which can in turn be used to control a control system.

The control system is configured to provide display data on the basis of the acquired measurement data which can be displayed on a first graphics unit on the at least one display means. The first graphics unit is advantageously a pictorial representation of a lung or of another organ that is affected by the ventilator. More advantageously, the first graphics unit comprises an animated representation.

By means of the inventive construction and the connection of the elements, the operator is provided with a reliable ventilator which is easy to operate. The visibility is substantially improved for the operator compared with known ventilators because of the configurable screen. Here, a qualitative as well as quantitative appreciation by the operator of the ventilator is guaranteed at all times.

Preferably, the control unit comprises a control logic module or a graphic logic module, wherein the acquired measurement data are processed on the one hand in the control logic module or in the graphic logic module, or both in the control logic module and also in the graphic logic module. Furthermore, the control logic module and the graphic logic module may form a common unit which is integrated into the ventilator, for example. Furthermore, at least the control logic module may also serve to control the ventilator.

All of said features in themselves guarantee a stable operation of the ventilator, thereby providing high reliability in use thereof.

Advantageously, both the control logic module and also the graphic logic module each have a computing unit, so that the acquired data in each module can be processed and provided for further use.

Preferably, the configurable screen is a touch-sensitive screen, whereupon it can serve not only to output display data, but also as an input means. Touch-sensitive screens of this type are also known as touchscreens. Further non-limiting examples of this type of touch-sensitive screens are touchpads or smart phones, smart watches, which are directly or indirectly connected to the ventilator or parts thereof, for example via a wireless connection such as, for example, Bluetooth® or WLAN.

Preferably, a sensor for acquiring at least one region of the at least one display means is provided, whereupon an unexpected change on the display can easily be detected and if necessary, appropriate measures such as alarms, internal instrument tests, can be initiated. If, for example, the display fails, the user may be sent a message, for example on their pager or mobile phone, so that they can react promptly.

Furthermore, the sensor can monitor visual displays and thus, in addition to monitoring through the control system, can provide an additional, independent monitoring unit. This further enhances the safety of the ventilator.

Advantageously, this sensor is adjacent to and more advantageously disposed directly on the at least one display means, whereupon a simple constructional configuration is made possible. This sensor may be a component of the sensor system linked to the ventilator.

The method in accordance with the invention for controlling said ventilator is characterized by the following steps:

Acquiring at least two items of measurement data with the sensor system (step a)) and subsequently transmitting the acquired measurement data from the sensor system to the ventilator or to the control system (step b)).

Subsequently, receiving at least one of the acquired items of measurement data from the ventilator or from the control system (step c)).

Advantageously, subsequently, individual items of the acquired measurement data are processed by the ventilator or by the control system.

Subsequently, display data are provided which are produced on the basis of at least individual items of received measurement data (step d)).

Consequently, at least individual items of display data are displayed in a first animated representation of a respiratory gas on a first graphics unit of the at least one display means (step e)), whereupon the display data can be visually and intuitively appreciated by an operator.

In this manner, a method for controlling a ventilator is provided which has a high reliability. The operator of a ventilator (in particular the medical professional) is notified at least visually of changes in respiratory parameters in the ventilator, whereupon they can then react so that the patient being ventilated by the ventilator does not come to harm.

The individual display data may contain acquired measurement data, received measurement data or processed measurement data or any combination of acquired measurement data, received measurement data and processed measurement data. The term “processed measurement data” includes any mathematical or logical modification to the acquired measurement data. The acquired measurement data are acquired by the sensor system. Alternatively or in addition, the acquired measurement data are input by the operator of the ventilator on an input means.

The at least individual items of display data are advantageously represented by at least individual geometrical elements, whereupon visibility for the operator is additionally enhanced and the operator is visually sensitized to the individual items of display data.

In this manner, advantageously, each individual item of display data which describes the same respiration parameters is displayed with geometrical elements having identical geometrical properties and each individual item of display data which describes different respiratory parameters is displayed with geometrical elements with different geometrical properties.

The term “geometrical property” of a geometrical element should be understood to mean the shape, color and size of the element. In this regard, the elemental shape should be understood to mean a two-dimensional shape (circle, triangle, ellipse, polygon, and so forth) or a three-dimensional shape (sphere, pyramid, cone, cube, and so forth).

Advantageously, the display data in step d) is provided by means of the control system, whereupon the display data can be provided easily. As an example, the display data are provided in the control logic module.

Alternatively or in addition, display data are provided by means of a graphic logic module with which, in addition to a graphical display of the display data, a simple display of the display data is obtained and thus the operator can quickly detect malfunctions in the ventilator and also can react to them quickly.

Advantageously, the graphic logic module is a component of the display means, whereupon a simplified construction in the ventilator is guaranteed.

Advantageously, the measurement data received (step c)) from the control logic module of the control system are transmitted to the graphic logic module, by means of which the measurement data can easily be graphically displayed on the at least one display means.

Advantageously, at least individual items of transmitted measurement data from the graphic logic module are processed in order to provide display data. With this feature, the quantity of measurement data to be processed can be reduced.

Preferably, in step c), the received measurement data can be divided in the control system into categories of measurement data, wherein at least individual items of measurement data from at least one measurement data category are transmitted to the graphic logic module. In this manner, the quantity of measurement data which has to be processed by the graphic logic means can be reduced.

Advantageously, all of the measurement data from the at least one measurement data category is transmitted to the graphic logic module, thereby ensuring improved statistics in the quantity of measurement data to be processed subsequently.

Alternatively or in addition, at least individual items of measurement data from a measurement data category are transmitted to the at least one display means, thereby preventing an incorrect display of display data, for example.

Advantageously, all of the measurement data from the one measurement data category is transmitted to the at least one display means, thereby ensuring improved statistics in the visualized display data.

Preferably, at least individual items of display data are displayed with a further representation which can be animated in the at least one display means, thereby guaranteeing an improvement in the visual receptivity of the ventilator operators as regards specific respiratory parameters on the display means and they can more easily make the necessary, and in particular the right decisions. In this regard, in addition to the first graphics unit, further animated displays may be depicted which in addition are readily visually discernible by the operator.

Advantageously, at least individual items of the display data are displayed with at least further individual geometrical elements in the further animatable representation in the at least one display means, whereupon the visual distinguishability of the individual items of display data and thus of the individual respiratory parameters by the operator of the ventilator can be promoted.

Advantageously, at least one geometrical property of an individual geometrical element in one of the animatable representations is modified, so that the operator of the ventilator can observe the variation with time of the individual items of display data and thus of the individual respiratory parameters. In this manner, the operator of the ventilator can react quickly and easily to modifications. Furthermore, this provides an enhanced reliability of the ventilator.

Preferably, at least individual items of the display data are displayed on the further animatable representation in the first graphics unit of the at least one display means, whereupon the visual distinguishability of the individual items of display data by the ventilator operator is further improved.

Preferably, at least one computing unit of the control logic module or of the graphic logic module calculates at least one distribution of the respiratory gas with the aid of the received individual items of measurement data. In this manner, incorrect measurement data are statistically eliminated, and thus an improved set of measurement data is generated.

Alternatively or in addition, in addition to the calculated distribution of the respiratory gas, with the aid of the received individual items of measurement data, a disposition of the respiratory gas is calculated with which, in addition to a statistical evaluation of the measurement data, a disposition of the respiratory gas which is known to the operator of the ventilator may also be calculated.

Preferably, the distribution of the respiratory gas is displayed in at least the first animatable representation of the respiratory gas, whereupon the operator of the ventilator is quickly made aware of a disruption to the respiratory procedure or a malfunction of the ventilator.

Alternatively or in addition, the disposition of the respiratory gas is displayed in at least the first animatable representation of the respiratory gas, whereupon the operator of the ventilator is easily made aware of a malfunction of the ventilator.

Advantageously, the animatable representation of the respiratory gas is displayed with the aid of the at least one geometrical element, whereupon the operator of the ventilator who has been trained on the individual geometrical elements can react quickly.

Preferably, at least the first graphics unit of the at least one display means may be modified at least in regions, whereupon, for example, the operator can actively interface with the graphics unit.

Advantageously, a modification to at least one region of the first graphics unit generates a control value which is subsequently transmitted to the control system. This feature ensures that the operator of the ventilator can interface directly with the control system via the at least one graphics unit, therefore ensuring simple operation of the ventilator as well as a high reliability.

Preferably, the at least one first animatable representation of a respiratory gas is depicted in the at least one first graphics unit of the display means, whereupon the at least one first animatable representation of the respiratory gas is represented with the aid of at least individual items of display data. In this manner, an improved visual sensitization of the operator of the ventilator to the display data is ensured.

Preferably, the at least individual displayed geometrical element, which represents at least individual items of display data, describes at least one respiratory parameter, whereupon the operator can be trained visually as regards each individual geometrical element and can assign the at least one respiratory parameter to the geometrical element.

Advantageously, the at least individual displayed geometrical element describes at least one respiratory parameter from the group formed by oxygen parameters, carbon dioxide parameters and lung pressure parameters, wherein at least individual displayed geometrical elements are represented by at least one characteristic geometrical property. This feature means that a visual display of the respiratory parameters on the at least one display means is possible.

Preferably, an exhausted fraction of respiratory gas can be distinguished from a fresh fraction of respiratory gas in the at least one first animatable representation of the respiratory gas, by displaying the respective fractions of respiratory gas using different individual geometrical properties. In this manner, the operator of the ventilator obtains a rapid overview and can react quickly to malfunctions in the ventilator.

Preferably, measurement data of individual respiratory parameters are displayed in a manner which can be animated, whereupon the operator can react easily to any malfunction in the ventilator.

Alternatively or in addition, difference values for measurement data from different respiratory parameters are displayed in an animatable manner, whereupon in addition, different respiratory parameters can be changed on the ventilator.

Preferably, at least one further animatable representation is provided in the display means, which comprises at least a portion of the first graphics unit, wherein the at least a portion of the first graphics unit with its geometrical properties is highlighted. In this manner, the visual perception of the operator of the ventilator is sensitized to individual particularly important respiratory parameters.

Advantageously, the at least one portion of the first graphics unit is highlighted with these geometrical properties in regions, whereupon the operator of the ventilator is directed to an important region in at least a portion of the graphics unit.

Preferably, the at least one display means has a further graphics unit which includes a chart with a graphical element, for example a line, wherein the graphical element represents at least a variation of display data with time, wherein the display data represent at least one respiratory parameter. In this manner, the variation with time of a respiratory parameter can be observed retrospectively.

Advantageously, the graphical element in the chart is matched with at least one geometrical property of the corresponding geometrical element in one of the animatable representations, whereupon the orientation of the operator of the ventilator towards the at least one display means is improved.

Preferably, the further graphics unit has a bar chart for animatable representation of a parameter of the ventilator. In this manner, the operator is presented with a particularly relevant parameter in a graphical manner.

In particular, the bar chart has an upper limit and a lower limit, wherein typically, a maximum allowable value or a minimum allowable value for the parameter can be represented, and thus a risk zone for the parameter can be depicted for the operator.

Preferably, the ventilator is linked to a further display means which contains individual items of display data from the ventilator, wherein the at least one display means and the further display means are advantageously spaced apart from each other, whereupon the operator of the ventilator obtains information regarding the ventilator from various instruments and can also react quickly, even over a distance.

Preferably, the ventilator is connected to a tomographic measuring device, wherein at least individual items of measurement data from the tomographic measuring device are transmitted to the control system and are received at least in the control system, which can be taken into consideration in one of the animatable representations in the first graphics unit. In this manner, it is possible to improve the calculation of the distribution or the disposition of the respiratory gases in the first graphics unit.

Advantageously, the tomographic measuring device is an electrical impedance tomography measuring device, whereupon a particularly accurate determination of measurement data can be carried out for the ventilator and a particularly accurate calculation of the distribution or of the disposition of the respiratory gases in the first graphics unit is made possible.

Further advantages, features and details of the invention will become apparent from the following description which describes exemplary embodiments of the invention with reference to the drawings.

The list of reference numerals as well as the technical content of the patent claims and the figures form part of the disclosure. The figures are described together and comprehensively. Identical reference numerals indicate identical components; reference numerals with different indices indicate components with identical or similar functions.

### BRIEF DESCRIPTION OF THE DRAWINGS

FIG. 1 shows a first embodiment of the ventilator with a first animatable representation of a respiratory gas in accordance with the invention in a lung as the first graphics unit on a display means in a perspective view.

FIG. 2 shows the animatable representation of the respiratory gas in a lung as the first graphics unit in accordance with FIG. 1 upon inhalation, in a perspective view.

FIG. 3 shows the animatable representation of the respiratory gas in a filled lung as the first graphics unit in accordance with FIG. 1, in a perspective view.

FIG. 4 shows a further animatable representation of the respiratory gas in a lung as the first graphics unit in accordance with FIG. 1, in a perspective view.

FIG. 5 shows a further animatable representation of the respiratory gas in a lung as the first graphics unit in accordance with FIG. 1, in a further perspective view.

FIG. 6 shows a further animatable representation of the respiratory gas in a lung as the first graphics unit in accordance with FIG. 1, in a further perspective view.

FIG. 7 shows a further animatable representation of the respiratory gas in a lung as the first graphics unit in accordance with FIG. 1, in a further perspective view.

FIG. 8 shows a further animatable representation of the respiratory gas in a lung as the first graphics unit in accordance with FIG. 1, in a further perspective view.

FIG. 9 shows a further animatable representation of the respiratory gas in a lung as the first graphics unit in accordance with FIG. 1, in a further perspective view.

FIG. 10 shows a further animatable representation of the respiratory gas in a lung as the first graphics unit in accordance with FIG. 1, in a further perspective view.

FIG. 11 shows a further animatable representation of the respiratory gas in a lung as the first graphics unit in accordance with FIG. 1, in a further perspective view.

FIG. 12 shows a further animatable representation in a bar chart as the second graphics unit in accordance with FIG. 1, in a further perspective view.

### DETAILED DESCRIPTION OF THE PREFERRED EMBODIMENT

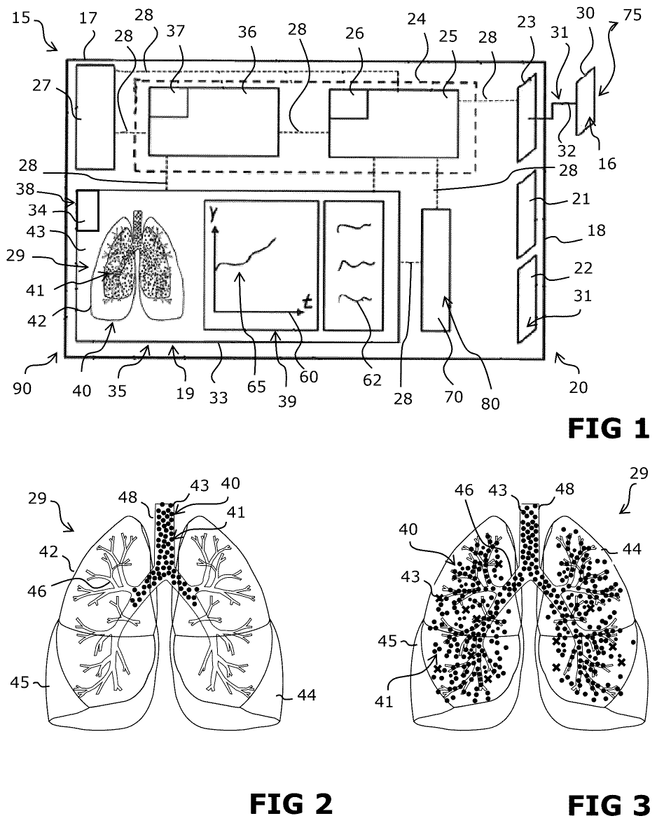

FIG. 1 shows a ventilator 15 with a housing 17 on the housing wall 18 of which a connecting means 20 is disposed. A first display means 35 is disposed on the housing front 19. A control system with a control logic module 25 which comprises a computing unit 26 (for example a processor) and a storage means 27 are positioned in the housing 17 of the ventilator 15, along with a graphic logic module 36 which comprises a computing unit 37 (for example a processor). The control logic module 25 and the graphic logic module 36 are electrically connected to each other with the aid of data lines 28. In addition to supply connections 21 (such as current supply, internet connection, gateway connection and so forth), the connecting means 20 comprises a ventilation tube connection 22 as well as a plurality of sensor connections 23. Measurement data 31 acquired from the external sensor system 30 are transmitted to the control logic module 25 with the aid of conventional data connections 32 (cable, WLAN, Bluetooth®, and so forth) and, for example, A/D transformers (not shown) by means of the sensor connection 23 and the ventilation tube connection 22. In the control logic module 25, the acquired measurement data 31 are either processed directly and/or transmitted to the graphic logic module 36 and at least a portion thereof is stored in the storage means 27. The control logic module 25 is connected to the first display means 35 via data lines 28. A sensor 34 is provided on the first display means 35, which captures a region 38 of the display means 35. The first display means 35 has a configurable screen 33 with a first graphics unit 29 and a second graphics unit 39. The first graphics unit 20 comprises the animatable representation 40 of the respiratory gas 41 in the lung 42. The second graphics unit 39 displays a chart 60 (y, t chart) with which the variation with time of one of the items of display data 65 as well as the numerical details of individual items of display data 62 are displayed. The respiratory parameters 16 are displayed directly on the first display means 35 with the aid of the display data 62, 65 or will initially be processed in the computing unit 26 of the control logic module 25 and subsequently displayed as display data 65 on the first display means 35 and/or in the first graphics unit 29 with an appropriate distribution of the respiratory gas 41 (homogeneous or non-homogeneous distribution, Gaussian distribution, exponential distribution, and so forth). The housing front 19 furthermore has an input means 70 which is electrically connected to the control logic module 25 with the aid of data lines 28, and with which the operator 90 (for example medical professionals) of the ventilator 15 can input individual respiratory parameters 16 as well as patient parameters 80.

As an example, the ventilator 15 is connected to a tomographic measuring device (not shown) which transmits its measurement data 31 to the control logic module 25. These measurement data 31 contribute to the processing of respiratory parameters 16, wherein the computing unit 26 of the control logic module 25 uses it, for example, to calculate the distribution of the respiratory gas 43 in the lung 42 and subsequently enters the result thereof into the animatable representation 40 of the respiratory gas 41. An electrical impedance tomography measuring device is envisaged as the preferred tomographic measuring device.

The following FIGS. 2 to 11 show the various embodiments of the animatable representations 40, 50 of the respiratory gas 41 in the lung 42 in the first graphics unit 29, wherein the lung 42 consists of two sections of the lung, or lobes, 44, 45, which are linked together by the trachea 48 as well as the respective bronchial tubes 46. The respiratory gas 41 is composed of a plurality of components (for example oxygen, nitrogen, noble gases, carbon dioxide, and so forth), which are displayed on the first display means 35 with the aid of a variety of respiratory parameters 16 or display data 62, 65 as well as being represented by geometrical elements 43 which can be distinguished from each other. In this regard, the geometrical elements 43 are shown in a two-dimensional manner (for example circles, dashes, triangles, and so forth) or in a three-dimensional manner (spheres, bars, pyramids, and so forth). The geometrical elements 43 of the animatable representation 40 are displayed in various manners which depend on the embodiment of the ventilator 15 in accordance with the invention, in different elemental sizes, elemental shapes as well as elemental colors.

As an example, all of the respiratory parameters 16 or display data 62, 65 are displayed in the animatable representation 40 as circles which differ in their diameter.

In the animatable representation 40, in the healthy state, the geometrical elements 43 are distributed homogeneously and completely when the lung 42 is filled, starting from the trachea 48, via the bronchial tubes 46 into the two sections of the lung 44, 45 (FIG. 2 and FIG. 3). Here, the parameters of oxygen concentration, provided by the control logic module 25 with the aid of the measured inhaled oxygen (FiO2_mess), the established fraction of the inhaled oxygen (FiO2_set), and the measured oxygen saturation (SpO2), the carbon dioxide parameter, which is measured with the aid of a CO₂ sensor, as well as the parameter for the lung overpressure, which is provided by the control logic module 25 with the aid of the measured proximal pressure and the tracheal pressure, can respectively be characterized with the same geometrical element 43, but with different elemental colors and/or elemental sizes in the animatable representation 40 (FIG. 3).

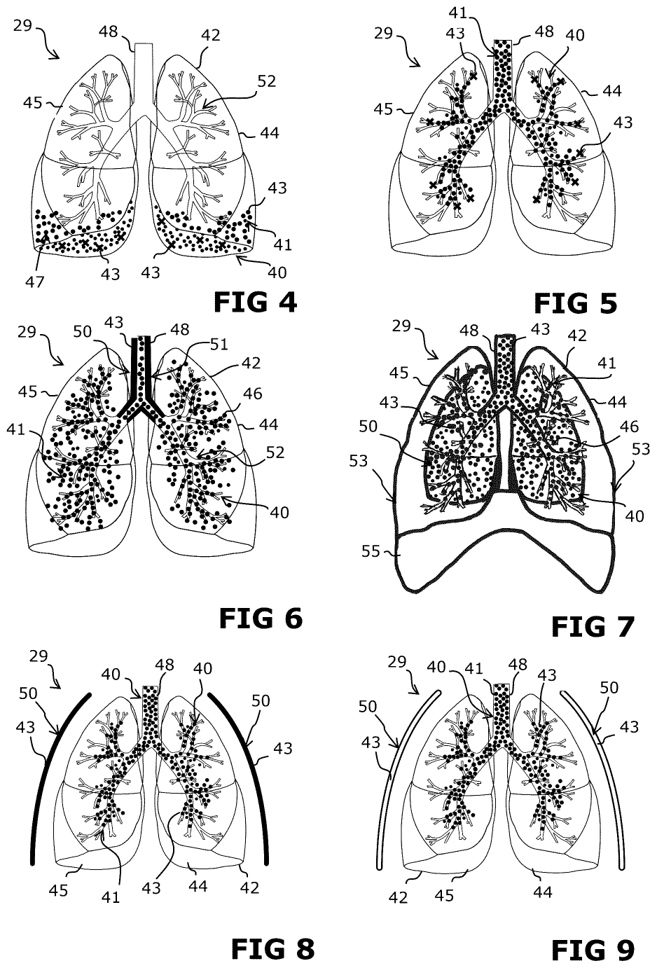

FIG. 4 shows the animatable representation 40 of the respiratory gas 41 in the lung 42, wherein in the case of a hyperinflatory lung 42, the respiratory gas 41 collects in the lower region of the lung 47 of the sections of the lung 43, 44. By measuring the automatic positive end-expiratory pressure parameter (auto-PEEP) continuously with the aid of a suitable sensor system 30, an increasing measurement value for the auto-PEEP is evaluated by the control logic module 25 and is depicted in the animatable representation 40. To this end, in the animatable representation 40, the exhausted respiratory gas fraction (for example the saturated carbon dioxide fraction or the exhausted oxygen fraction) and the fresh respiratory gas fraction (freshly supplied respiratory gases 41) are shown in different shades of grey with the same geometrical element 43.

When measuring the auto-PEEP, those regions of the lung 47 (for example pulmonary alveoli) which still contain residual respiratory gas 41 can be depicted with the aid of the animatable representation 40 of the respiratory gas 41. These pulmonary alveoli on the bronchial tubes 46 are respectively depicted with the aid of a geometrical element 43 (a circle) (FIG. 5).

As can be seen in FIG. 6, a restriction in the trachea 48 can be animated with the aid of the further representation 50. Here, the trachea wall 51 as well as the bronchial wall 52 are shown with thicker lines and in a color that is different from that for a healthy lung. In addition, the respiratory gas 41 in this animated representation 40 is disposed such that the geometrical elements can be positioned one behind the other in a line.

FIG. 7 shows a lung 42 with an increased compliance of the lung. This is determined by the control logic module 25 using the compliance as a respiratory parameter 16 and is shown by means of a combination of the first animatable representation 40 of the respiratory gas 41, which shows a spatially restricted distribution of the geometrical elements 43 in the sections of the lung 44, 45 and of the further animatable representation 50, which shows up as colored highlighting of the wall of the lobe of the lung 53. In this regard, the degree of lung lobe compliance is represented by the width of the colored highlighting of the wall of the lobe of the lung 53. In addition, the diaphragm 55 is shown in a different color, which is processed in the control logic module 25 when spontaneous breathing of the patient is measured and is shown in the further animatable representation 50.

FIG. 8 and FIG. 9 show the animatable representation 50 of an oesophageal pressure measurement in the lung 42, wherein the conclusions drawn from the oesophageal pressure measurement are displayed with the aid of the geometrical elements 43 in the form of measuring bars outside the lung 42. In this regard, the control logic module 25 processes measurement data 31 for the lung pressure measurement and the interpleural pressure measurement in the lung 42, in which, for example, a difference value for the measurement data 31 is produced, which is then shown as display data 62, 65 in the animatable representation 50 in the form of measuring bars with different colors.

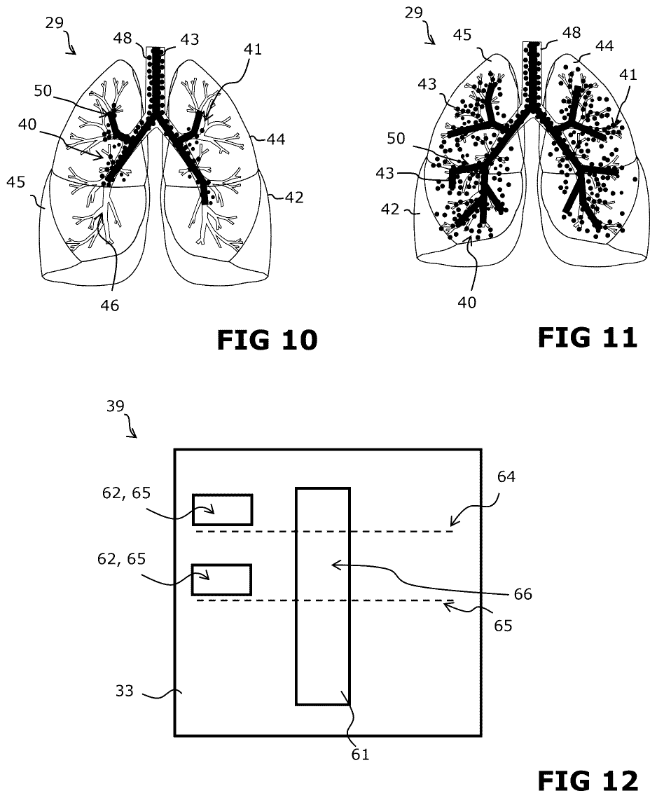

FIG. 10 and FIG. 11 show the representation of a ratio of the PEEP value to the difference between the PEEP value and the pressure upon inspiration (PINSP) which are processed by the control logic module 25 and then are depicted in the lung with the aid of the animatable representation 40. If the values for the ratio are raised, the elemental color of the geometrical elements 43 changes and are highlighted in color in the lung 42 with the aid of the measuring bar. In a preferred embodiment, the method for controlling a ventilator 15 in accordance with the invention comprises the following steps.

After the sensor system 30 has acquired measurement data 31, the measurement data 31 are delivered to the ventilator 15 and its control system 24 and are then processed by the ventilator 15 by storing the measurement data 31 in the storage means 27 and/or by processing in the control system 24. There, measurement data 31 are either combined with data from the storage means 27 or processed in a manner such that they are displayed as display data 62, 65. In the processing process, the computing unit 26 of the control logic module 25 or the computing unit 37 of the graphic logic module 36 quantitatively and qualitatively combines the measurement data 31 (optionally with historical measurement data) with the input respiratory parameters 16. After combining the respiratory parameters 16, the control logic module 25 assigns those respiratory parameters 16 which are shown in one of the animatable representations 40, 50 of the respiratory gas 41 to a geometrical element 43 and displays it in the first graphics unit 29 with the associated elemental shape, elemental color and elemental size. At the same time, the control logic module 25 or the graphic logic module 36 determines the variation of the same respiratory parameters 16 with time and displays them in the chart 60 with the same colors or with the same shape or elemental size. At the same time, display parameters 62 are displayed on the second graphics unit.

As an example, the opening up of collapsed regions of the lung (lung recruitment) can be depicted as an animation. In a first step in this regard, a controllable respiratory pressure (for example the PEEP) is slowly raised, whereupon its variation with time in chart 60 as well as the associated geometrical element 43 are shown in the same color in the animatable representation 40. Next, ventilation is stopped, the respiratory pressure (for example the PEEP) is slowly reduced again, whereupon its variation with time is displayed in the chart 60 and also the geometrical element 43 is displayed in the animatable representation 40 in the same color, but can be distinguished from the first step. These two steps are repeated until the greatest difference (hysteresis) is established in the two steps. The respiratory pressure (for example the PEEP) determined thereby is subsequently passed from the control logic module 25 to the control system 24 and is given as the new control value in the ventilator 15. When there is a change (possibly an unforeseen malfunction), the operator 90 can interface directly with the control system on the ventilator 15 by changing one of the items of display data 62, 65 in the first graphics unit 29. This generates a control value which is then transmitted to the control system 40. The geometrical elements 43 described above which represent the individual respiratory parameters 16 or display parameters 65 in the lung 42 can differ in their shape, size as well as color from each other, depending on the embodiment.

FIG. 12 shows an animatable representation of a parameter 66 in a bar chart 61 as the second graphics unit 39 on the configurable screen 33. The bar chart 61 has an upper limit 64 and a lower limit 65. As an example, the bar chart 61 represents a particularly relevant parameter 66 of the ventilator such as, for example, the ventilation performance or the overall performance or the transpulmonary performance. The maximum allowable value or minimum allowable value for the parameter 66 is shown by the upper limit 64 or the lower limit 65, whereupon the risk zone for the parameter 66 can be shown to the operator 90. At the same time, further significant display data 62, 65 such as, for example, the dead volume or the respiratory rate, can be shown on the upper limit 64 and the lower limit 65. The upper limit 64 and the lower limit 65 can be determined for the patient 75 to be ventilated, whereupon a variation in the parameter 66 can be shown as an animation. As an example, a change to the parameter 66 can be shown with an animated change to the representational color. The representation of the second graphics unit 39 together with the representation of the first graphics unit 29 can be shown as an animation.

### REFERENCE LIST

15 ventilator
16 respiratory parameter
17 housing
18 housing wall
19 housing front
20 connecting means
21 supply connections
22 ventilation tube connection
23 sensor connections
24 control system
25 control logic module
26 computing unit for 25
27 storage means
28 data lines
29 first graphics unit
30 measurement data
31 sensor system
32 data link
33 configurable screen
34 sensor
35 first display means
36 graphic logic module
37 computing unit for 36
38 region
39 second graphics unit
40 animatable representation
41 respiratory gas
42 lung
43 geometrical element
44 section of lung
45 section of lung
46 bronchial tubes
47 lung region
48 trachea
50 further animatable representation
51 trachea wall
52 bronchial tube wall
53 lobe wall
55 diaphragm
60 chart (y, t chart)
61 bar chart
62 display data (digital)
63 lower limit
64 upper limit
65 display data (digital)
66 parameter
70 input means
75 patient
80 patient parameter
90 operator

1-30. (canceled)

31. A system for providing animated ventilation information for a patient, the system comprising:
at least one processor configured to receive respiratory parameter data associated with a patient ventilation provided by a ventilator; and
at least one display device communicatively coupled to the at least one processor and configured to provide an animatable representation of the respiratory parameter data, wherein the animatable representation is configured to display:
(a) a first pictorial representation of lung lobes,
(b) a second pictorial representation of bronchial walls within the lung lobes, and
(c) a third pictorial representation of a trachea configured to be animated separately from at least a portion of the second pictorial representation and with one or more of a different color or different line thickness than the second pictorial representation, the one or more of the different color or line thickness being provided based on the respiratory parameter data being indicative of restriction.

32. The system of claim 31, wherein the animatable representation is configured to display one or more of:
(a) the first pictorial representation of the lung lobes having geometric elements representative of respiratory gas provided in a lower region of the lung lobes based on the respiratory parameter data being indicative of hyperinflatory lungs,
(b) the first pictorial representation of the lung lobes having a colored highlighting of a periphery of the lung lobes indicative of a degree of lung lobe compliance, or
(c) a fourth pictorial representation of a diaphragm based on the respiratory parameter data being indicative of spontaneous breathing.

33. The system of claim 31, wherein the at least one display device comprises a touch-sensitive screen.

34. The system of claim 31, wherein:
the at least one display device is directly connected to the ventilator; or
the at least one display device is indirectly connected to the ventilator via a wireless connection.

35. The system of claim 31, wherein the at least one display device is configured to provide at least one graphical element comprising a representation of a variation of at least one respiratory parameter with time.

36. The system of claim 31, wherein the at least one display device is configured to provide at least one graphical element providing different colors of a bar as a representation of at least one respiratory parameter.

37. The system of claim 31, wherein the at least one display device is configured to provide at least one graphical element comprising numerical details for individual items of display data.

38. The system of claim 31, wherein the at least one processor is operably coupled to the ventilator and is configured to control the ventilator based on one or more of
(a) a control value based on an operator input comprising a change to at least one item of display data at the at least one display device, or
(b) measurement data for the respiratory parameter data.

39. The system of claim 31, wherein the at least one processor is configured to send a message to at least one user device indicative of a change in displayed information at the at least one display device.

40. The system of claim 31, wherein the at least one processor is operably coupled to the ventilator and is configured to determine an automatic positive end-expiratory pressure (auto-PEEP).

41. A system for providing animated ventilation information for a patient, the system comprising:
at least one processor configured to receive respiratory parameter data associated with a patient ventilation provided by a ventilator; and
at least one display device communicatively coupled to the at least one processor and configured to provide an animatable representation of the respiratory parameter data,
wherein the animatable representation is configured to display at least one of:
(a) a first pictorial representation of first lung lobes having geometric elements representative of respiratory gas provided in a lower region of the first lung lobes based on the respiratory parameter data being indicative of hyperinflatory lungs, or
(b) a second pictorial representation of second lung lobes having a colored highlighting of a periphery of the second lung lobes indicative of a degree of lung lobe compliance.

42. The system of claim 41, wherein the animatable representation is configured to display one or more of:
(a) a third pictorial representation of bronchial walls within the first lung lobes and/or the second lung lobes with a fourth pictorial representation of a trachea configured to be animated separately from at least a portion of the third pictorial representation with one or more of a different color or different line thickness based on the respiratory parameter data being indicative of restriction, or
(b) a fifth pictorial representation of a diaphragm that is provided based on the respiratory parameter data being indicative of spontaneous breathing.

43. The system of claim 41, wherein the at least one display device comprises a touch-sensitive screen.

44. The system of claim 41, wherein:
the at least one display device is directly connected to the ventilator; or
the at least one display device is indirectly connected to the ventilator via a wireless connection.

45. The system of claim 41, wherein the at least one display device is configured to provide at least one graphical element comprising a representation of a variation of at least one respiratory parameter with time.

46. The system of claim 41, wherein the at least one display device is configured to provide at least one graphical element providing different colors of a bar as a representation of at least one respiratory parameter.

47. The system of claim 41, wherein the at least one display device is configured to provide at least one graphical element comprising numerical details for individual items of display data.

48. The system of claim 41, wherein the at least one processor is operably coupled to the ventilator and is configured to control the ventilator based on one or more of:
(a) a control value based on an operator input comprising a change to at least one item of display data at the at least one display device, or
(b) measurement data for the respiratory parameter data.

49. The system of claim 41, wherein the at least one processor is configured to send a message to at least one user device indicative of a change in displayed information at the at least one display device.

50. The system of claim 41, wherein the at least one processor is operably coupled to the ventilator and is configured to determine an automatic positive end-expiratory pressure (auto-PEEP).

51. A system for providing animated ventilation information for a patient, the system comprising:
at least one processor configured to receive respiratory parameter data associated with a patient ventilation provided by a ventilator; and
at least one display device communicatively coupled to the at least one processor and configured to provide an animatable representation of the respiratory parameter data,
wherein the animatable representation is configured to display a first pictorial representation of a diaphragm when the respiratory parameter data indicates spontaneous breathing of the patient.

52. The system of claim 51, wherein the animatable representation is configured to display one or more of:
(a) a second pictorial representation of first lung lobes having bronchial walls within the lung lobes with a third pictorial representation of a trachea configured to be animated separately from at least a portion of the bronchial walls with one or more of a different color or different line thickness based on the respiratory parameter data being indicative of restriction,
(b) a fourth pictorial representation of second lung lobes having geometric elements representative of respiratory gas provided in a lower region of the second lung lobes based on the respiratory parameter data being indicative of hyperinflatory lungs, or
(c) a fifth pictorial representation of third lung lobes having a colored highlighting of a periphery of the third lung lobes indicative of a degree of lung lobe compliance.

53. The system of claim 51, wherein the at least one display device comprises a touch-sensitive screen.

54. The system of claim 51, wherein:
the at least one display device is directly connected to the ventilator; or
the at least one display device is indirectly connected to the ventilator via a wireless connection.

55. The system of claim 51, wherein the at least one display device is configured to provide at least one graphical element comprising a representation of a variation of at least one respiratory parameter with time.

56. The system of claim 51, wherein the at least one display device is configured to provide at least one graphical element providing different colors of a bar as a representation of at least one respiratory parameter.

57. The system of claim 51, wherein the at least one display device is configured to provide at least one graphical element comprising numerical details for individual items of display data.

58. The system of claim 51, wherein the at least one processor is operably coupled to the ventilator and is configured to control the ventilator based on one or more of:
(a) a control value based on an operator input comprising a change to at least one item of display data at the at least one display device, or
(b) measurement data for the respiratory parameter data.

59. The system of claim 51, wherein the at least one processor is configured to send a message to at least one user device indicative of a change in displayed information at the at least one display device.

60. The system of claim 51, wherein the at least one processor is operably coupled to the ventilator and is configured to determine an automatic positive end-expiratory pressure (auto-PEEP).
